(19) United States
(12) Patent Application Publication
(10) Pub. No.: US 2012/0232377 A1
(43) Pub. Date: Sep. 13, 2012

(54) SURGICAL NAVIGATION FOR REVISION SURGICAL PROCEDURE

(76) Inventor: Eric W. Nottmeier, Atlantic Beach, FL (US)

(21) Appl. No.: 13/416,984

(22) Filed: Mar. 9, 2012

Related U.S. Application Data
(60) Provisional application No. 61/451,499, filed on Mar. 10, 2011.

(51) Int. Cl. A61B 6/00; A61B 17/70; A61B 6/03; A61B 5/055; A61B 8/00; A61B 6/02

(52) U.S. Cl. 600/411; 600/424

(57) ABSTRACT

Surgical navigation for revision surgery includes temporality coupling a reference arc to a surgical object that was substantially permanently affixed to a bone of a patient during a preceding surgery. The reference arc includes a navigation tracker configured to communicate with a navigation system. A surgical instrument is coupled to a respective navigation tracker that is also configured to communicate with the navigation system. A known relationship between the navigation tracker of the reference arc and the navigation tracker of the surgical instrument is used to determine a spatial characteristic of the surgical instrument, such as an position or orientation of the surgical instrument. The spatial characteristic is rendered upon a display unit.

### CROSS-REFERENCES TO RELATED APPLICATIONS

This application claims priority to, and the benefit of U.S. Application Ser. No. 61/451,499, filed on Mar. 10, 2011, titled “Surgical Navigation For Revision Surgical Procedure,” the entire contents of which is incorporated herein by reference.

### STATEMENT REGARDING FEDERALLY SPONSORED RESEARCH

Not Applicable.

### BACKGROUND

1. Field of the Invention

Embodiments generally relate to methods, systems, and devices for surgical navigation and, more particularly, methods, systems, and devices for surgical navigation for a revision surgical procedure.

2. Description of the Related Art

The success of a surgical procedure often relies on its accuracy. For example, malpositioning of a pedicle screw during spinal surgery can cause neurological injury to the patient (e.g., human or animal). Surgical navigational technology can improve surgical accuracy because it enables surgeons to visualize a patient's anatomy and to track locations of anatomical landmarks and surgical instruments. Using current surgical navigational technology, a reference arc is attached to the bony anatomy of the patient, which allows a computer to track and accommodate for any changes in the patient's position. In spinal surgical procedures in which surgical navigational technology is used, this reference arc is typically clamped directly to the spine on a dorsal bony protuberance called the spinous process. Previous surgeries on a patient, however, alter anatomy and in some cases the patient's spinous processes may have been removed making it difficult to apply the reference arc.

It would, therefore, be desirable to have methods, systems, and devices for use in image-guided revision surgical procedures.

### SUMMARY

In one embodiment, a method for surgical navigation during revision surgery of a patient includes temporarily coupling a reference arc to a surgical object that was substantially permanently affixed to a bone of the patient during a preceding surgery. A distal end of the reference arc is coupled to a first navigation tracker configured to communicate with a navigation system. A surgical instrument is used to conduct the revision surgery. The surgical instrument includes a second navigation tracker that is configured to communicate with the navigation system. The second navigation tracker of the surgical instrument has a known relationship with the navigation tracker of the reference arc. The navigation system calculates a spatial characteristic, such as a position or orientation, of the surgical instrument using the know relationship.

In certain embodiments, a method for surgical navigation during revision surgery includes making an incision in a patient to expose at least a portion of a vertebral column of the patient, wherein the revision surgery occurs subsequent to an initial surgery on the vertebral column. A proximal end of a reference arc is temporarily coupled to lateral connector that was substantially permanently coupled to the vertebrae of the patient during the initial surgery. A distal end of the reference arc is coupled to a first navigation tracker configured to communicate with a navigation system. A surgical instrument is used to conduct the revision surgery. The surgical instrument includes a second navigation tracker configured to communicate with the surgical navigation system. The second navigation tracker has a known relationship with the first navigation tracker. The navigational system calculates a spatial characteristic of the instrument from the known spatial relationship.

In certain embodiments, a method for surgical navigation during revision surgery includes receiving, at a processor of a computing device during revision surgery upon a vertebral column of a patient, data from a first navigation tracker of a reference arc. The reference arc is temporarily coupled to a surgical object that was substantially permanently affixed to a vertebrae of the patient during a preceding surgery. The first navigation tracker is configured to communicate with a navigation system. The processor also receives data from a second navigation tracker of a surgical instrument. The second navigation tracker is configured to communicate with the navigation system. The second navigation tracker has a known relationship with the first navigation tracker. The processor calculates a spatial characteristic of the surgical instrument using the known relationship and renders the spatial characteristic on a display unit.

An exemplary advantage of certain embodiments includes obtaining a more accurate known relationship between navigation trackers due to a stable coupling between the reference arc and the bone of the patient. It is yet another advantage of certain embodiments to couple a reference arc to a rod which is connected to a pedicle screw that is substantially permanently affixed to a bone of a patient such that the surgical object is imaged using an imaging device.

### BRIEF DESCRIPTION OF THE DRAWINGS

Embodiments will become more apparent from the detailed description set forth below when taken in conjunction with the appended claims and the drawings, in which like elements bear like reference numerals.

FIG. 1A is a side view of a human vertebral column;

FIG. 1B is a top view of a vertebrae including a pair of substantially, permanently affixed pedicle screws;

FIGS. 2A and 2B are each a radiograph of a human vertebral column after an initial surgery in which pedicle screws were substantially permanently affixed to a plurality of vertebrae of the vertebral column and rods are connected to the pedicle screws;

FIG. 2C is a schematic of a human vertebral column that has undergone an initial surgery in which pedicle screws are substantially permanently affixed to a plurality of vertebrae and rods are connected to the pedicle screws and one another via lateral connectors;

FIG. 3 is a front perspective view of an exemplary navigation system;

FIG. 4 is a diagram of an exemplary navigation system;

FIG. 5A is a schematic of a vertebral column prior to an initial surgery;

FIG. 5B is a schematic of a vertebral column after an initial surgery but prior to a revision surgery;

FIGS. 6A-6B are a schematics of exemplary reference arcs that are coupled to at least one of a pedicle screw, a rod, or a lateral connecter;

FIG. 7 illustrates a flow chart of an exemplary method for surgical navigation for revision surgery; and

FIG. 8 illustrates a flow chart that is a continuation of the method illustrated in FIG. 7.

### DETAILED DESCRIPTION OF THE INVENTION

Reference throughout this specification to “one embodiment,” “an embodiment,” or similar language means that a particular feature, structure, or characteristic described in connection with the embodiment is included in at least one embodiment. Thus, appearances of the phrases “in one embodiment,” “in an embodiment,” “certain embodiments,” and similar language throughout this specification may, but do not necessarily, all refer to the same embodiment.

The described features, structures, or characteristics of various embodiments may be combined in any suitable manner. In the following description, numerous specific details are recited to provide a thorough understanding of embodiments. One skilled in the relevant art will recognize, however, that the invention may be practiced without one or more of the specific details, or with other methods, components, materials, and so forth. In other instances, well-known structures, materials, or operations are not shown or described in detail to avoid obscuring aspects of the various described embodiments.

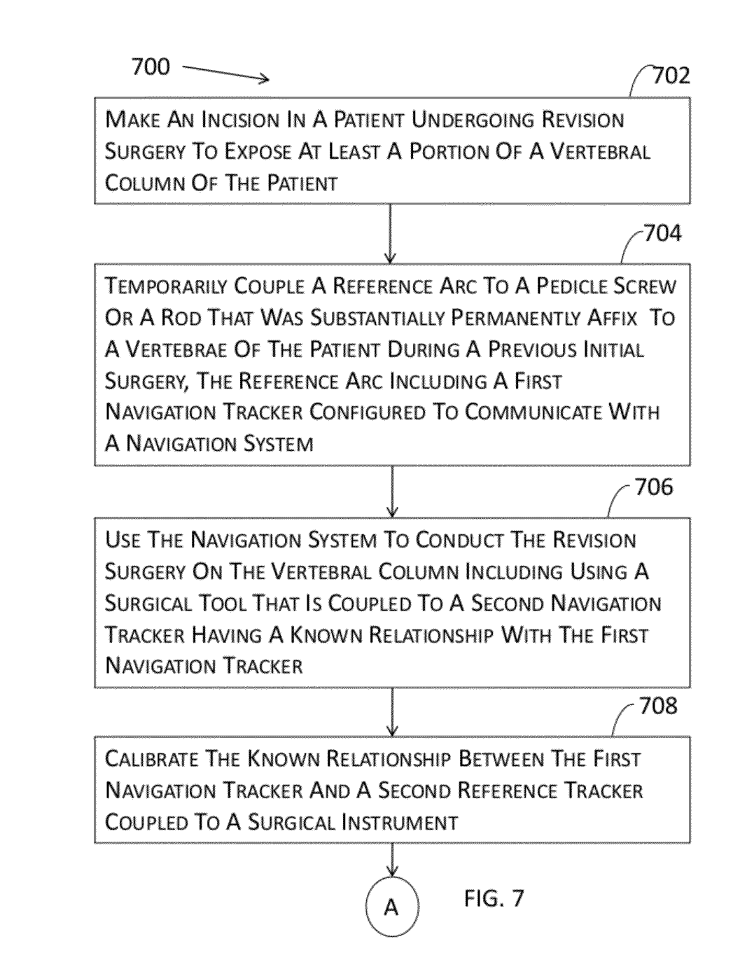

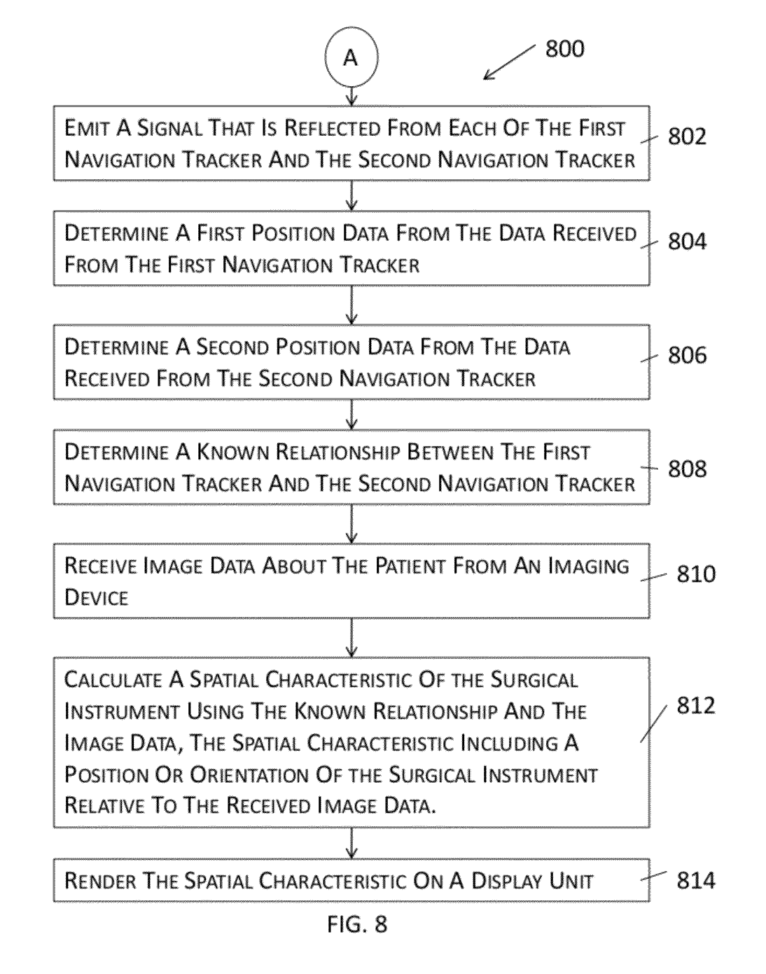

The schematic flow chart diagrams included are generally set forth as a logical flow-chart diagram (e.g., FIGS. 7 and 8). As such, the depicted order and labeled steps are indicative of an embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow-chart diagrams, they are understood not to limit the scope of the corresponding method (e.g., FIGS. 7 and 8). Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

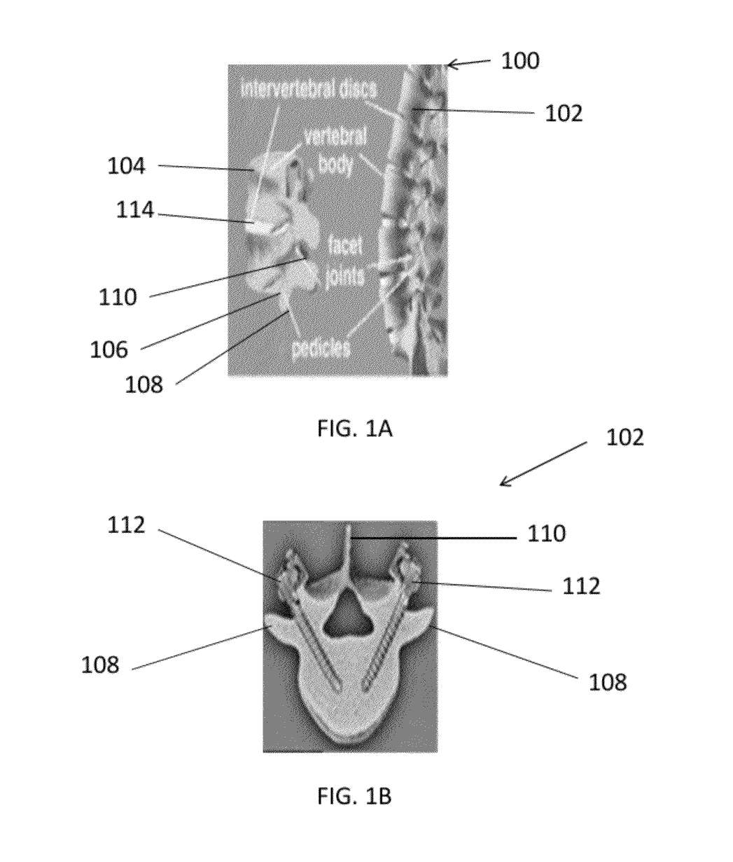

Referring to FIGS. 1A and 1B, a vertebral column 100 of a human patient includes twenty four articulating vertebrae 102 and nine fused vertebrae in the sacrum and coccyx (not shown). The anterior of the vertebrae 102 includes a vertebral body 104 that is formed by a pair of pedicles 106. The vertebrae also has two transverse processes 108 and a spinous process 110 at the posterior side. An intervertebral disc 114 lies between adjacent vertebrae in the vertebral column 100. Abnormalities in the vertebral column 100 that are congenital (e.g., scoliosis) or due to disease (e.g., herniated disc), trauma (e.g., whiplash), or aging (e.g., arthritis) may require corrective measures through surgical intervention.

In some instances, the surgical intervention includes substantially permanently affixing a surgical object, such as a pedicle screw, rod, lateral connector, or an implant, into a bone of the patient. FIG. 1B illustrates an instance in which the surgical object includes two pedicle screws 112 that are substantially permanently affixed to a vertebrae of a patient. Here, each pedicle screw 112 is implanted into a corresponding vertebral pedicle 106. The pedicle screw 112 may be, for example, a polyaxial pedicle screw that is made of biocompatible materials such as Titanium. In certain embodiments, pedicle screw 112 length range from about 30 mm to about 60 mm with a diameter range from about 4.0 mm to about 8.5 mm, for example.

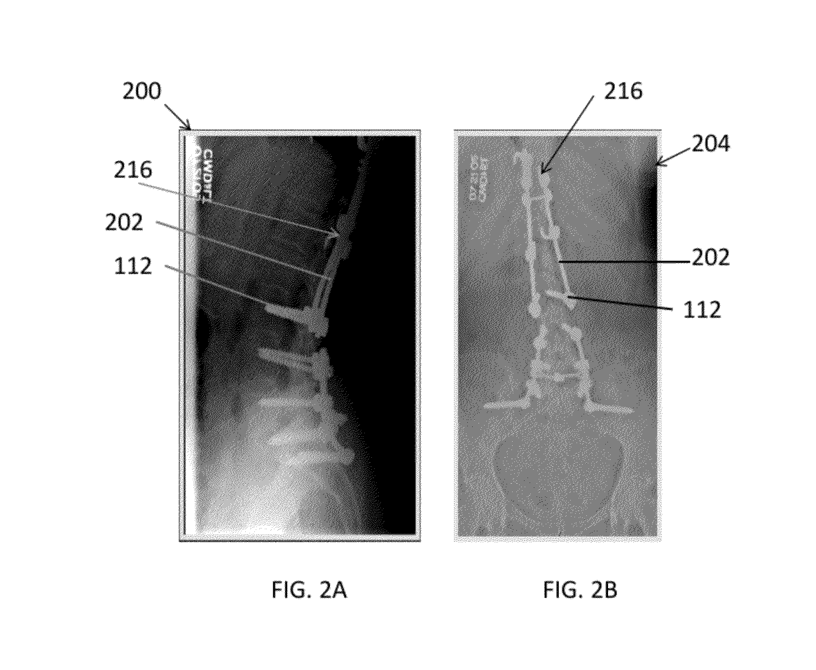

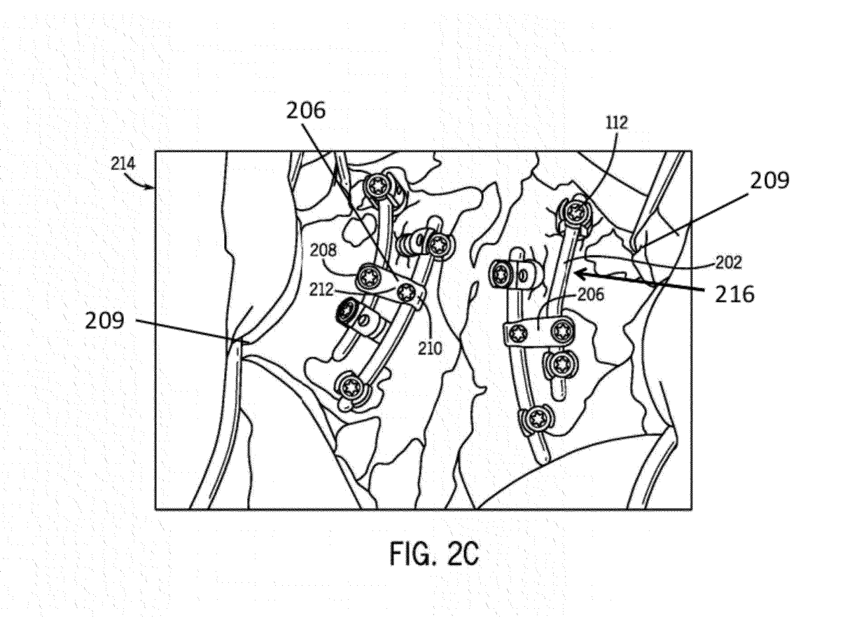

Referring to FIGS. 2A and 2B, radiographs 200 and 204 show sagittal plane and coronal plane images, respectively, of a patient that has undergone spinal surgery to correct a vertebral column 100 abnormality. The images of FIGS. 2A and 2B include images of the surgical object 216 (e.g., a pedicle screw 112 and a rod 202), that was substantially permanently affixed to the vertebral column 100 of a patient during an initial surgery. Referring to FIG. 2C, a schematic 214 illustrates a patient that has undergone spinal surgery. The soft tissue 209 of the patient is pulled back to expose the surgical object affixed to the boney structure of the patient. In certain embodiments, the surgical object 216 includes a pedicle screw 112, a rod 202, a lateral connector 206, or a combination thereof. In certain embodiments, the lateral connector 206 of the surgical object has a first end 208, a second, opposite end 210, and a body 212.

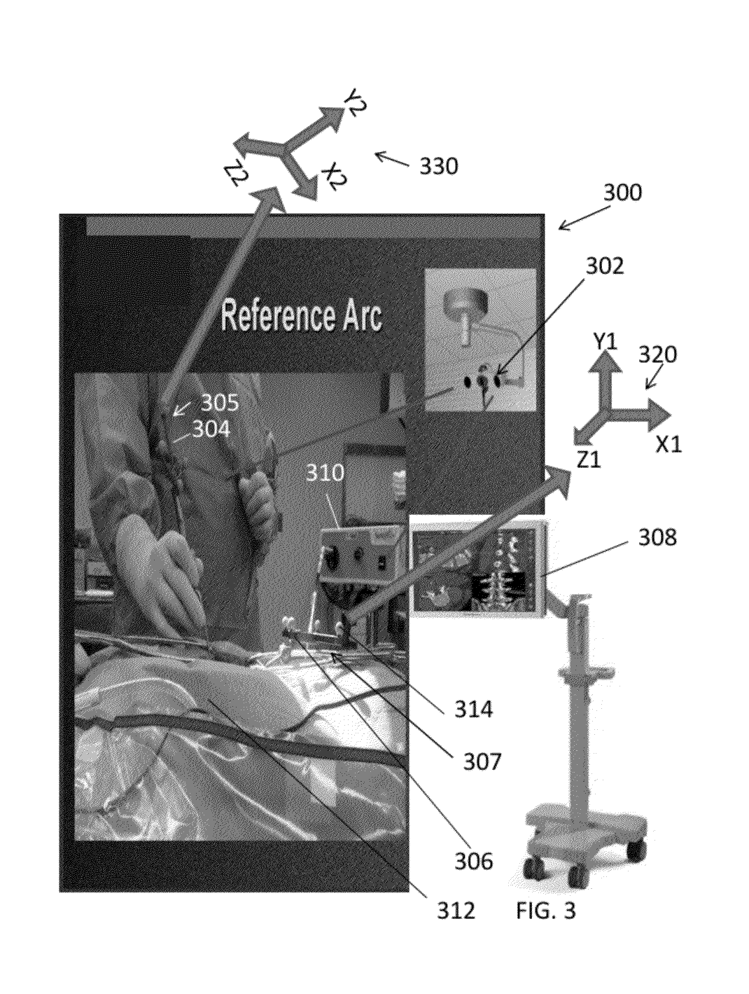

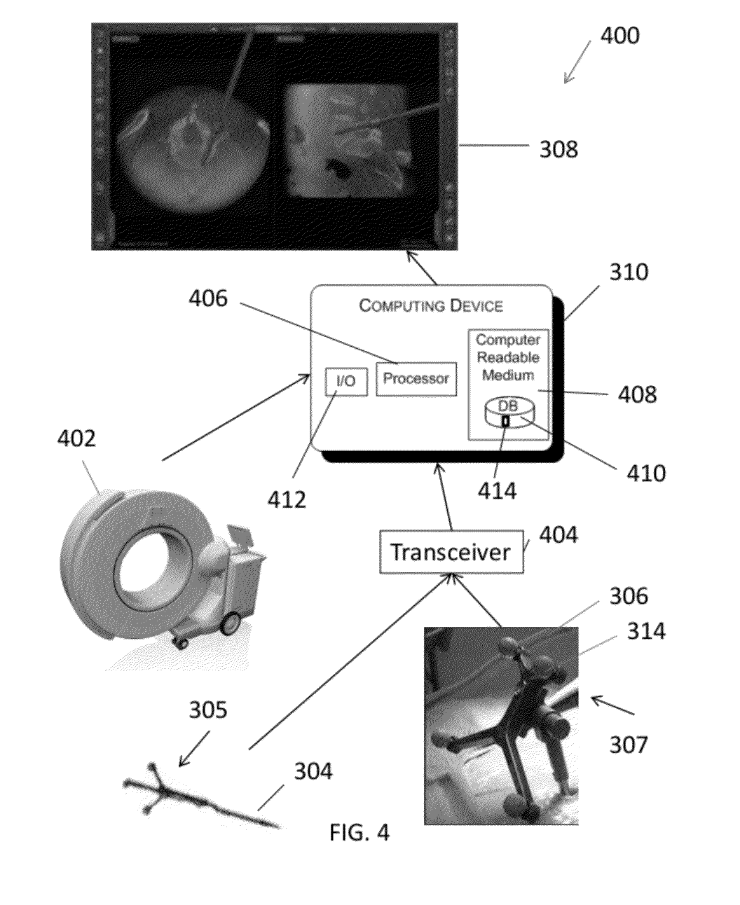

Surgical navigation systems assist health care providers, such as surgeons, to visualize internal structures of patients and the relative location or orientation of their surgical instruments for presurgical planning or for guiding or performing surgery. Referring to FIGS. 3 and 4, exemplary surgical navigation systems 300 and 400 are illustrated, respectively. In FIG. 3, a patient 312 is positioned on an operating table or bed. The surgical navigation systems 300 and 400 each include a reference arc 306, a surgical instrument 304, a computing device 310, and a display 308. Although a single reference arc 306, surgical instrument 304, computing device 310, and display 308 are illustrated in FIGS. 3 and 4, any number of the forgoing may be included in the navigation systems 300 or 400. Each of the reference arc 306 and the surgical instrument 304 includes a corresponding navigation tracker 305 and 307, respectively, which is configured to communicate with a transceiver that is, in turn, configured to communicate with the computing device 310.

In certain embodiments, the surgical navigation system uses an optical Infrared tracking system, an electromagnetic tracking system, or a combination thereof, for example. Other forms of tracking systems are also contemplated. To illustrate, the tracking systems may be any one of a passive optical system, an active optical system, a magnetic based system, an inertial navigation system, any combination of the forgoing, and the like. In FIG. 3, the surgical navigation system 300 includes a camera array 302 adapted to track navigation trackers 305 and 307 by receiving Infrared signals emitted (e.g., via active markers) or reflected (e.g., via “inactive” or “passive” markers 314) from the navigation trackers 305 and 307. The camera array 302 sends the received Infrared signals to the computing device 310. In the example of FIG. 3, the camera array 302 includes three charge-coupled device cameras that are mounted in a stationary position relative to one another. The camera array 302 has a known orientation data that is also sent to the computing device 310.

In certain embodiments, the computing device 310 uses the known orientation data of the camera array 302 and the Infrared signals received from the navigation tracker 307 of the reference arc 306 and the navigation tracker 305 of the surgical instrument 304 to determine a relationship between the two navigation trackers 307 and 305. The determined relationship is then used to determine a spatial characteristic of the surgical instrument 304, such as its position or orientation of the surgical instrument 304. To illustrate, the computing device 310 uses the orientation data of the camera array 302 and the Infrared signals received from the plurality of Infrared markers affixed to the navigation tracker 307 of the reference arc 306 to determine a coordinate system 320 (X1 axis, Y1 axis, and Z1 axis with an origin at their intersection) for the navigation tracker 307 of the reference arc 306. The computing device 310 also uses the orientation data of the camera array 302 and the Infrared signals received from the plurality of Infrared markers affixed to the navigation tracker 305 of the surgical instrument 304 to determine a coordinate system 330 (X2 axis, Y2 axis, and Z2 axis with an origin at their intersection) for the navigation tracker 305 of the surgical instrument 304. The relationship between the coordinate system 320 of the reference arc 306 and the coordinate system 330 of the surgical instrument 304 is mathematically determined by a translation and rotation of one coordinate system into the other. Consequently, the position and orientation of the surgical instrument 304 relative to the reference arc 306 is tracked using the determined, now known, relationship between the navigation tracker 307 of the reference arc 306 and the navigation tracker 305 of the surgical instrument 304. In FIG. 4, the surgical navigation system 400 includes a transceiver 404 capable of transmitting and receiving electromagnetic signals from electromagnetic navigation trackers 305 and 307.

Referring to FIGS. 3 and 4, the navigation systems 300 and 400 each depict a computing device 310 that is configured to communicate with the navigation trackers 305 and 307. In certain embodiments, the computing device 310 is an article of manufacture such a special purpose machine, such as a server, a mainframe computer, a desktop, a laptop, and/or a tablet, having one or more processors (e.g., a Central Processing Unit, a Graphical Processing Unit, and/or a microprocessor) that is configured to execute an algorithm (e.g., a computer readable program code or software) to receive data, transmit data, store data, or perform methods. In certain embodiments, the computing device 310 includes a non-transitory computer readable medium having a series of instructions, such as computer readable program steps deposited therein. In certain embodiments, the non-transitory computer readable medium includes one or more data repositories.

In certain embodiments, the data repositories are one or more hard disk drives, tape cartridge libraries, optical disks, or any suitable volatile or nonvolatile storage medium, storing one or more databases, or the components thereof, in a single location or in multiple locations, or as an array such as a Direct Access Storage Device (DASD), redundant array of independent disks (RAID), virtualization device, . . . etc. To illustrate, the data repository is structured by a database model, such as a relational model or a hierarchical model). In certain embodiments, the computing device 310 includes wired and wireless communication devices which employ various communication protocols including near field (e.g., “Blue Tooth”) and far field communication capabilities.

By way of example, the computing device 310 of FIG. 4 is shown as a special purpose computer, including a processor 406, a non-transitory computer readable medium 408, an input/output means 412 (e.g., a keyboard, a mouse, a touch screen, a receiver, a transceiver, a transmitter, or a printer) and a data repository DB 410. The processor 406 accesses executable code stored on the non-transitory computer readable medium 408, and executes one or more instructions to electronically receive data from the navigation tracker 305 and/or 307, for example. In certain embodiments, the surgical navigation system 300 or 400 may also include an imaging device, such as imaging device 402. Here, the computing device 310 utilize data from the imaging device 402, such as a Computed Tomography Scanner, a Magnetic Resonance Imaging Scanner, an X-ray machine, an ultrasound device, a Positron Emission Tomography Scanner, or a fluoroscope, to generate a model or image of an anatomical region of interest of the patient. In FIG. 4, the exemplary imaging device 402 is shown as an O-arm® surgical imaging device by Medtronic, Inc. The data about the anatomical region of interest is uploaded into a computing device 310. The data is then processed, such as through data fusion techniques, to create a dataset representing a two-dimensional or three-dimensional model of the anatomical region of interest. The model produces a near geometric replica of both the normal and abnormal tissue or structures of the region of interest. The image may also include landmark features that help align the model with data collected during surgery, such as a location of the surgical instrument 304 or location of a previously implanted surgical object.

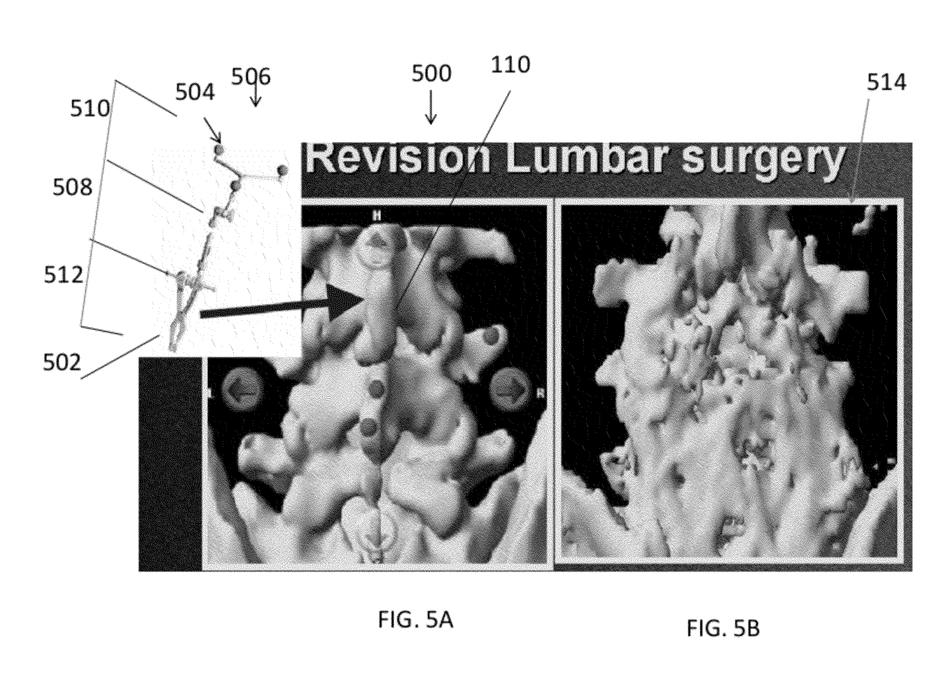

Referring to FIGS. 5A and 5B, a reference arc 506 includes a body 508 having a distal end 510 and an opposite, proximal end 512. The proximal end 512 is coupled to a connective adaptor, such as a clamp 502, and the distal end 510 may be coupled to a navigation tracker 504. In the example shown in FIG. 5A, the clamp 502 is configured to temporality couple to a healthy spinous process 110, anchoring it to a vertebrae. Here, the clamp 502 temporarily couples with the bone in a relatively stable manner such that it has a low probability of movement during the initial surgery.

Such anatomical anchors are not present during revision surgery. Revision surgery corrects an area of the anatomy that was operated upon during a previous surgery. Referring to FIG. 5B, an initial surgery has disfigured the vertebral column 514 of a patient such that the spinous process 110, for example, can no longer act as a stable anatomical anchor for the reference arc 506. If the reference arc 506 is clamped to a bone of the disfigured vertebral column 514, the probability of slippage or movement during the revision surgery is highly increased and, in turn, the risk of malpositioning of pedicle screws 112 during the revision surgery is also increased.

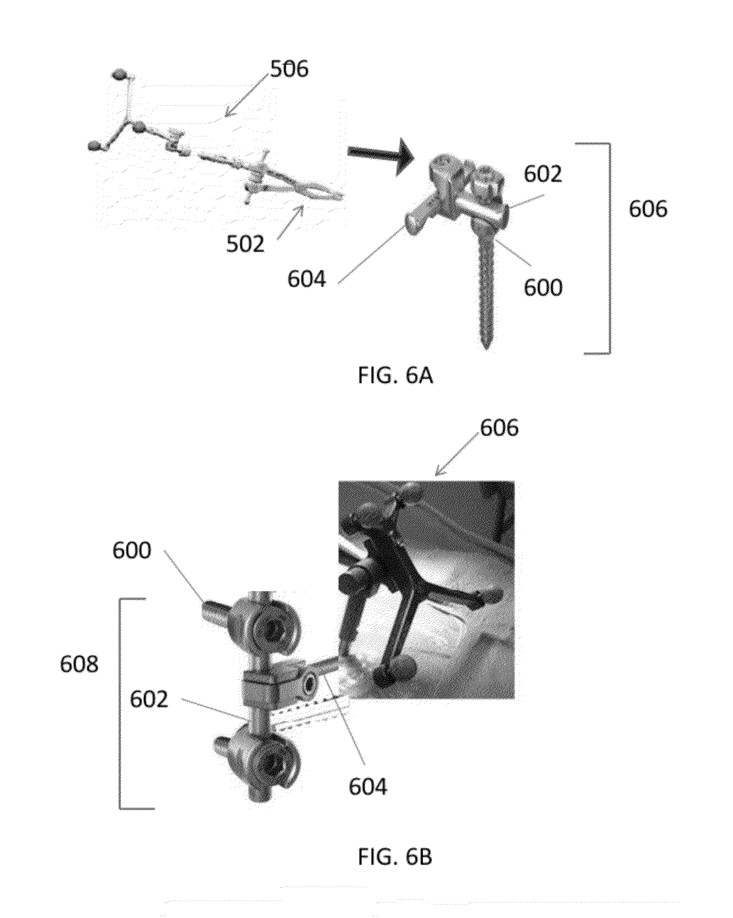

Referring to FIGS. 6A and 6B, the reference arcs 506 and 606 are each temporarily coupled to surgical objects 606 and 608, respectively, that was substantially permanently affixed to a bone of the patient during the preceding surgery. For example, the clamp 502 is temporarily coupled to a portion of a pedicle screw 600 (e.g., pedicle screw 112 of FIG. 2A-2C) protruding from the bone, the rod 602 (e.g., rod 202 of FIG. 2A-2C), the lateral connector 604 (e.g., lateral connector 206 of FIG. 2A-2C), or a combination thereof, that was substantially permanently affixed to the bone during the initial surgery.

In certain embodiments, the clamp 502 is coupled to the lateral connector 604 that was coupled to one or more rods 602 that were each coupled to one or more corresponding pedicle screws 600 during the initial surgery. For example, the reference arc 606 is coupled to one end of the lateral connector 604 while the opposite end of the lateral connector 604 is coupled to the rod 602 (FIG. 6B). In another example, the reference arc 606 is coupled to a body of the lateral connector 604 located between the two ends of the lateral connector 604. Here, each end of the lateral connector 604 is coupled to a corresponding rod 602 and the corresponding rod 602 is coupled to one or more pedicle screws 600.

Referring to FIG. 7, a flow chart illustrates an exemplary method 700 for surgical navigation for revision surgery in a patient (e.g., human or animal). At a step 702, an incision is made in a patient undergoing revision surgery to expose at least a portion of a vertebral column of the patient. At a step 704, a reference arc (e.g., reference arc 306 of FIG. 3 or reference arc 506 of FIG. 5) is temporarily coupled to a surgical object, such as a rod, which is, in turn, coupled to a pedicle screw (e.g., pedicle screw 600 of FIG. 6A) that was substantially permanently affixed to a vertebrae of the patient during an initial surgery.

As stated previously the reference arc includes a navigation tracker configured to communicate with a navigation system (e.g., navigation system 300 of

FIG. 3 and/or navigation system 400 of FIG. 4). At step 706, the surgical instrument (e.g., surgical instrument 304 of FIG. 3) having a navigation tracker is used to conduct the revision surgery. As previously stated, the navigation tracker of the surgical instrument has a known relationship with the navigation tracker of the reference arc. In certain embodiments, the relationship between the navigation tracker of the surgical instrument and the navigation tracker of the reference arc is calibrated at step 708. Additionally, the reference arc will move with the patients anatomy if the operating room table height is adjusted up or down which enables the computer to account for the change in 3D space of the patient's anatomy.

Referring to FIG. 8, the method 700 of FIG. 7 continues as method 800 for surgical navigation for revision surgery is depicted. At step 802, a transceiver emits a signal that is, in turn, reflected from a plurality of inactive markers affixed to each of the navigation tracker of the reference arc and the navigation tracker of the surgical instrument. For example, the processor 406 of the computing device 310 of FIG. 4 facilitates the emission of a signal that is detected by a receiver communicatively connected to the processor 406. At step 804, the processor of the computing device (e.g., 310 of FIG. 4) determines marker positions from the signals reflected from the inactive markers affixed to the navigation tracker of the reference arc. At step 806, the processor determines marker positions from the signals reflected from the inactive markers affixed to the navigation tracker of the surgical instrument. At step 808, the processor uses the marker positions for each of the navigation trackers of the reference arc and surgical instrument to determine a known relationship between the navigation tracker of the reference arc and the navigation tracker surgical instrument.

At step 810, the processor receives image data about the region of interest of the patient from an imaging device (e.g., 402 of FIG. 4). At step 812, the processor accesses executable code stored in a non-transitory computer readable medium to execute instructions to calculate a spatial characteristic of the surgical instrument using the known relationship and the image data. To illustrate, the spatial characteristic is the position (e.g., within a coordinate system) or orientation (e.g., rotation) of the surgical instrument relative to the received image data or model of the anatomical region of interest. At step 812, the spatial characteristic is rendered on a display unit (e.g., 308 of FIG. 3).

The methods 700 and/or 800, or portions thereof, may be repeated periodically over a period of time during the revision surgery. For example, step 812, calculating the spatial characteristic of the surgical instrument, is periodically repeated over a period of time during the revision surgery.

Although the present invention has been described in detail with reference to certain embodiments, one skilled in the art will appreciate that the present invention can be practiced by other than the described embodiments, which have been presented for purposes of illustration and not of limitation. Therefore, the scope of the appended claims should not be limited to the description of the embodiments contained herein.

What is claimed is:

1. A method for surgical navigation during revision surgery on a patient, the method comprising:
(a) temporarily coupling, during a revision surgery on a patient, a proximal end of a reference arc to a surgical object that was substantially permanently affixed to a bone of the patient during a preceding surgery, wherein a distal end of the reference arc is coupled to a first navigation tracker configured to communicate with a navigation system;
and
(b) using a surgical instrument to conduct the revision surgery, wherein:
the surgical instrument includes a second navigation tracker configured to communicate with the navigation system;
the second navigation tracker has a known relationship with the first navigation tracker; and
the navigational system calculates a spatial characteristic of the surgical instrument using the known relationship.

2. The method of claim 1, further comprising calibrating the known relationship.

3. The method of claim 1 wherein the surgical object is selected from a group consisting of:
a pedicle screw;
a rod that is coupled to the pedicle screw;
a lateral connecter that is coupled to the rod; and
a combination thereof.

4. The method of claim 1 wherein the surgical object includes a lateral connecter that is coupled to a rod that is coupled to a pedicle screw.

5. The method of claim 1 wherein the navigation system includes an imaging device selected from the group consisting of:
a fluoroscope;
an X-ray machine;
an ultrasound device;
Positron Emission Tomography Scanner;
a Computed Tomography Scanner; and
a Magnetic Resonance Imaging Scanner.

6. The method of claim 1 wherein the first navigation tracker includes at least one of:
an active marker;
a passive marker; and
a combination thereof.

7. The method of claim 1 wherein the navigation system is selected from a group consisting of:
an infrared tracking system;
an electromagnetic tracking system; and
a combination thereof.

8. The method of claim 1 wherein the navigational system periodically calculates the spatial characteristic of the surgical instrument over a period of time during the revision surgery.

9. The method of claim 1 wherein the spatial characteristic is selected from a group consisting of:
a position of the surgical instrument;
an orientation of the surgical instrument; and
a combination thereof.

10. A method for surgical navigation during revision surgery on a patient positioned on an operating table, the method comprising:
(a) making an incision in a patient undergoing revision surgery to expose at least a portion of a vertebral column of the patient, wherein the revision surgery occurs subsequent to an initial surgery on the vertebral column;
(b) temporarily coupling a proximal end of a reference arc to lateral connector that was substantially permanently coupled to a vertebrae of the patient during the initial surgery, wherein a distal end of the reference arc is coupled to a first navigation tracker configured to communicate with a navigation system;
and
(c) using a surgical instrument to conduct the revision surgery, wherein:
the surgical instrument includes a second navigation tracker configured to communicate with the surgical navigation system;
the second navigation tracker has a known relationship with the first navigation tracker; and
the navigational system calculates a spatial characteristic of the instrument from the known spatial relationship.

11. The method of claim 10 wherein the lateral connector is coupled to a rod that is coupled to a pedicle screw that were each substantially permanently affixed to a vertebrae of the patient during the initial surgery.

12. The method of claim 10 wherein the navigation system includes an imaging device selected from the group consisting of:
a fluoroscope;
an X-ray machine;
an ultrasound device;
Positron Emission Tomography Scanner;
a Computed Tomography Scanner; and
a Magnetic Resonance Imaging Scanner.

13. The method of claim 10 wherein the first navigation tracker includes at least one:
an active marker;
a passive marker; and
a combination thereof.

14. The method of claim 10 wherein the navigation system is selected from a group consisting of:
an infrared tracking system;
an electromagnetic tracking system; and
a combination thereof.

15. The method of claim 10 wherein the navigational system periodically calculates the spatial characteristic of the surgical instrument over a period of time during the revision surgery.

16. A method for surgical navigation during revision surgery, the method comprising:
(a) receiving, at a processor of a computing device during revision surgery upon a vertebral column of a patient, data from a first navigation tracker of a reference arc that is temporarily coupled to a surgical object, wherein:
the first navigation tracker is configured to communicate with a navigation system; and
the surgical object was substantially permanently affixed to a vertebrae of the patient during a preceding surgery;
(b) receiving, at the processor, data from a second navigation tracker of a surgical instrument, wherein:
the second navigation tracker is configured to communicate with the navigation system; and
the second navigation tracker has a known relationship with the first navigation tracker;
(c) calculating, at the processor, a spatial characteristic of the surgical instrument using the known relationship; and
(d) rendering, using the processor, the spatial characteristic on a display unit.

17. The method of claim 16 further comprising using the processor to facilitate emission of a signal, wherein:
the signal is reflected off of a first plurality of markers coupled to the first navigation tracker and a second plurality of markers coupled to the second navigation tracker; and
the signals off of each of the first plurality of markers and the second plurality of markers is usable to determine the spatial characteristic.

18. The method of claim 16 further comprising:
receiving image data about the patient from an imaging device; and
using the known relationship to track at least one of a position and an orientation of the surgical instrument relative to the received image data, wherein the spatial characteristic includes at least one of the position and the orientation of the surgical instrument relative to the received image data.

19. The method of claim 16 further comprising:
periodically receiving, at the processor, the data from the first navigation tracker over a period of time during the revision surgery;
periodically receiving, at the processor, the data from the second navigation tracker over the period of time; and
periodically calculating the position of the surgical instrument based upon said received data from the first navigation tracker and said received data from the second navigation tracker.

20. The method of claim 16 wherein the surgical object is selected from a group consisting of:
a pedicle screw;
a rod that is coupled to the pedicle screw;
a lateral connecter that is coupled to the rod; and
a combination thereof.
